(19) United States
(12) Patent Application Publication
(10) Pub. No.: US 2013/0166015 A1
(43) Pub. Date: Jun. 27, 2013

(54) HYBRID AORTIC ARCH REPLACEMENT

(71) Applicant: Blayne A. Roeder, Lafayette, IN (US)

(72) Inventor: Blayne A. Roeder, Lafayette, IN (US)

(73) Assignee: Cook Medical Technologies LLC, Bloomington, IN (US)

(21) Appl. No.: 13/335,103

(22) Filed: Dec. 22, 2011

(51) Int. Cl. A61F 2/06; A61F 2/82

(52) U.S. Cl. 623/1.13; 623/1.35

(57) ABSTRACT

A hybrid prosthesis may include a distal graft portion including a tubular main body including a biocompatible material and a support structure. The distal graft portion may have proximal and distal ends and a main lumen extending therebetween. A tubular branch may extend from the main body of the distal graft portion and may have a branch lumen in fluid communication with the main lumen of the distal graft portion. The proximal graft portion may include a tubular body including a biocompatible material. The body of the proximal graft portion may be unstented and may have proximal and distal ends and a lumen extending therebetween. The distal end of the proximal graft portion may be attached to the proximal end of the distal graft portion. The lumen of the proximal graft portion may be in fluid communication with the main lumen of the distal graft portion.

### TECHNICAL FIELD

This disclosure relates to medical devices for implantation within the human or animal body for treatment of endovascular disease. More particularly, it relates to a hybrid prosthesis adapted for treating a thoracic aorta of a patient.

### BACKGROUND

Endovascular methods have been proposed for treatment of aneurysms of the aorta, particularly when an aneurysm is adjacent the aorta bifurcation. But when an aneurysm occurs higher up in the aorta, for example, in the region of the descending aorta adjacent the thoracic arch or in the ascending aorta, endovascular techniques for treating these aneurysms are somewhat more difficult because of the arched nature of the thoracic arch, the existence of major arteries in the region, and the proximity to the heart.

Generally, operations to treat aneurysms that include the ascending aorta or the arch have been done by open chest surgery. Such surgery generally involves surgical replacement of a portion of the aorta with a tubular prosthesis. The surgery is a high risk procedure. Two foremost reasons for the risk associated with the procedure are difficulty of accessing the site of treatment and the potential for neural ischemia.

The surgical Bentall technique has been demonstrated with some success for treating ascending aortic aneurysms. But the Bentall technique may be used only in patients able to tolerate a fully surgical technique and thus is not suitable for patients that may be intolerant of such an invasive procedure.

In dealing with aortic arch aneurysms, procedural risk is greatly increased by inclusion of the brachiocephalic vessels and the aorta distal to the arch. The difficulty of the procedure also may be exacerbated by the necessity to reconnect the left common carotid and left subclavian arteries to the tubular prosthesis after replacing a portion of the aorta. Although surgical techniques have been successfully demonstrated to repair arch aneurysms, such techniques are highly invasive and thus limited in utility, especially in high risk patients.

### SUMMARY

The present embodiments provide a hybrid prosthesis and systems and methods for facilitating deployment of such a prosthesis.

In one example, a hybrid prosthesis may include a distal graft portion and a proximal graft portion. The distal graft portion of the prosthesis may include a tubular main body including a biocompatible material and a support structure. The distal graft portion may have a proximal end, a distal end, and a main lumen extending between the proximal and distal ends. At least one tubular branch may extend from the main body of the distal graft portion of the prosthesis. The tubular branch may have a first end, a second end, and a branch lumen extending between the first and second ends. The branch lumen may be in fluid communication with the main lumen of the distal graft portion of the prosthesis. The proximal graft portion of the prosthesis may include a tubular body including a biocompatible material. The body of the proximal graft portion may be unstented and may have a proximal end, a distal end, and a lumen extending between the proximal and distal ends. The distal end of the proximal graft portion may be attached to the proximal end of the distal graft portion. The lumen of the proximal graft portion may be in fluid communication with the main lumen of the distal graft portion.

In another example, a system for the treatment of a vessel defect may include a hybrid prosthesis and an introducer. The prosthesis of the system may include a distal graft portion and a proximal graft portion. The distal graft portion of the prosthesis may include a tubular main body including a support structure. The distal graft portion may have a proximal end, a distal end, and a main lumen extending between the proximal and distal ends. At least one tubular branch may extend from the main body of the distal graft portion. The branch may have a first end, a second end, and a branch lumen extending between the first and second ends. The proximal graft portion of the prosthesis may extend from the distal graft portion of the prosthesis. The proximal graft portion may include a tubular body. The body of the proximal graft portion may be unstented and may have a proximal end, a distal end, and a lumen extending between the proximal and distal ends. The introducer of the system may include a central catheter. At least a portion of the distal graft portion of the prosthesis may be retained in a compressed configuration on the central catheter of the introducer for endoluminal delivery to a site of the vessel defect.

In yet another example, a method of treating a defect of a thoracic aorta of a patient may include making an incision in a thoracic arch of the patient. The incision may be positioned proximal to an ostium of a left subclavian artery of the patient. The method also may include introducing a distal graft portion of a hybrid prosthesis through the incision and into the thoracic aorta. The method also may include endoluminally positioning a distal end of the distal graft portion within a descending aorta of the patient and endoluminally positioning a branch of the distal graft portion within the left subclavian artery of the patient.

Other systems, methods, features, and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTIONS OF THE DRAWINGS

FIG. 1 is a perspective view of one embodiment of a hybrid prosthesis.

FIG. 2 is a perspective view of an alternative embodiment of a hybrid prosthesis.

FIG. 3 is a longitudinal cross sectional view of one embodiment of an introducer for a hybrid prosthesis.

FIG. 4 is a perspective view of one embodiment of an introducer for a hybrid prosthesis.

FIG. 5 depicts an initial configuration in the formation of a diameter reducing tie for a hybrid prosthesis.

FIG. 6 depicts the diameter reducing tie of FIG. 5 in its delivery configuration.

FIG. 7 is a schematic view of an aorta of a patient.

FIG. 8 illustrates one example of a method of delivering a distal graft portion of a hybrid prosthesis within an aorta of a patient.

FIG. 9 illustrates one example of a method of deploying a distal graft portion of a hybrid prosthesis within an aorta of a patient.

FIG. 10 illustrates one example of a method of placing a proximal graft portion of a hybrid prosthesis within an aorta of a patient.

### DETAILED DESCRIPTION OF THE DRAWINGS AND THE PRESENTLY PREFERRED EMBODIMENTS

The present disclosure relates to a hybrid prosthesis and systems and methods for facilitating deployment of such a prosthesis.

In the present disclosure, the term “proximal” refers to a direction that is generally closest to the heart during a medical procedure, while the term “distal” refers to a direction that is farthest from the heart during a medical procedure.

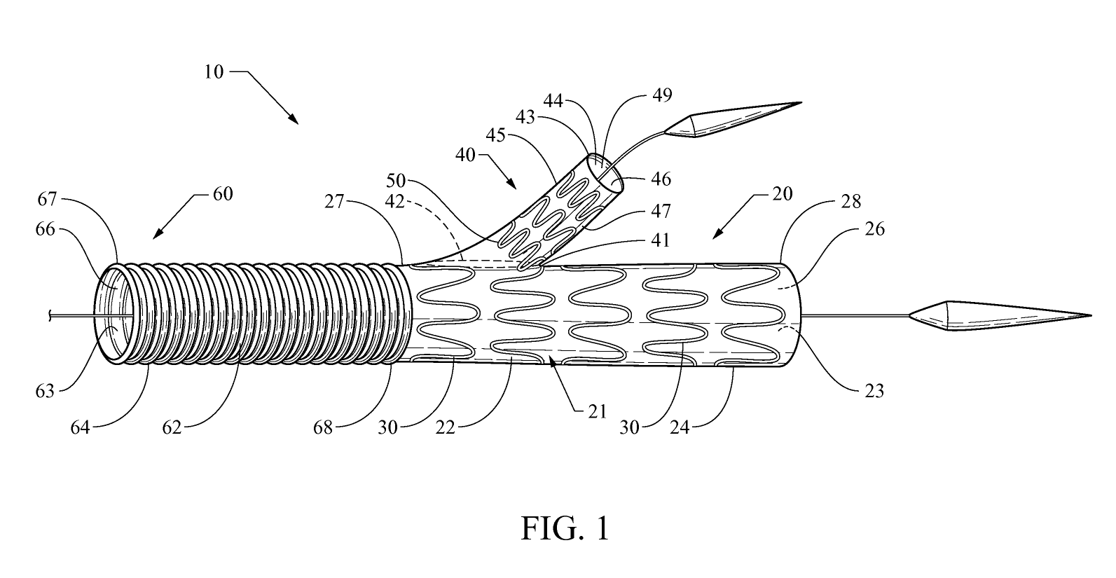

FIG. 1 depicts one example of a hybrid prosthesis 10. The hybrid prosthesis 10 may include a distal graft portion 20 and a proximal graft portion 60. As further described below, the distal graft portion 20 may be generally configured as a stent graft, while the proximal graft portion 60 may be generally configured as a surgical graft.

The distal graft portion 20 may include a main body 21 and a branch 40. The main body 21 may include a substantially tubular graft body 22 having an inner surface 23 and an outer surface 24. The graft body 22 may be configured as a generally tubular member having a substantially cylindrical shape. The inner surface 23 of the graft body 22 may define a main lumen 26 extending longitudinally within the main body 21 between a proximal end 27 and a distal end 28 thereof. The main lumen 26 may be suitable for passing fluid such as, for example, blood therethrough.

The main body 21 of the distal graft portion 20 also may include at least one support structure 30, such as a stent. The support structure 30 may be configured as a single, unitary structure or as a plurality of independent structures. The support structure 30 and/or various portions thereof may be disposed on the inner surface 23 and/or the outer surface 24 of the graft body 22. Multiple support structures 30 may be positioned at various locations along a length of the main body 21. A support structure 30 may be positioned near the distal end 28 of the distal graft portion 20 to seal the distal end against a wall of a body vessel. A support structure 30 also may be positioned near the proximal end 27 of the distal graft portion 20 and or the point of attachment between the main body 21 and the branch 40. This support structure may aid in fixing the prosthesis 10 in place with respect to the body vessel and/or supporting the branch 40 extending from the main body 21. The support structures 30 positioned near the distal end 28 and the proximal end 27 of the main body 21 may be positioned on the inner surface of the graft body 22.

The branch 40 of the distal graft portion 20 may have a first end 41 with a first end opening 42 and a second end 43 with a second end opening 44. The branch 40 may include a substantially tubular graft body 45 having an inner surface 46 and an outer surface 47. The graft body 45 may be configured as a generally tubular member having a substantially cylindrical shape. The inner surface 46 of the graft body 45 may define a branch lumen 49 extending longitudinally between the first end 41 and the second end 43 of the branch 40. The branch 40 also may include at least one support structure 50. The support structure 50 may include a single, unitary structure or a plurality of independent structures. The support structure 50 and/or various portions thereof may be disposed on the inner surface 46 and/or the outer surface 47 of the graft body 45. Multiple support structures 50 may be positioned at various locations along a length of the branch 40.

The branch 40 may extend from the main body 21. To that end, the graft body 45 of the branch 40 may be formed integrally with the graft body 22 of the main body 21. Alternatively, the branch 40 may be formed separately, and the first end 41 of the branch may be attached to the graft body 22. The branch 40 may extend from the main body 21 such that the distal graft portion 20 may have a generally Y shaped configuration as shown in FIG. 1. The branch 40 may be configured as a peripheral branch extending from a side of the main body 21 or a contralateral branch attached to a leg of a Y formed by the main body. The branch 40 may extend from the main body 21 at any angle with respect to the graft body 22. Preferably, the branch 40 may extend from the main body 21 at an acute angle as shown in FIG. 1. The branch 40 may extend from any point along a length of the main body 21 between the proximal end 27 and the distal end 28 of the main body. Preferably, the branch 40 may be attached to the main body 21 at a position that enables the branch to be aligned with a branching vessel as further described below. To that end, the branch 40 may be attached to the main body 21 near the proximal end 27 of the main body.

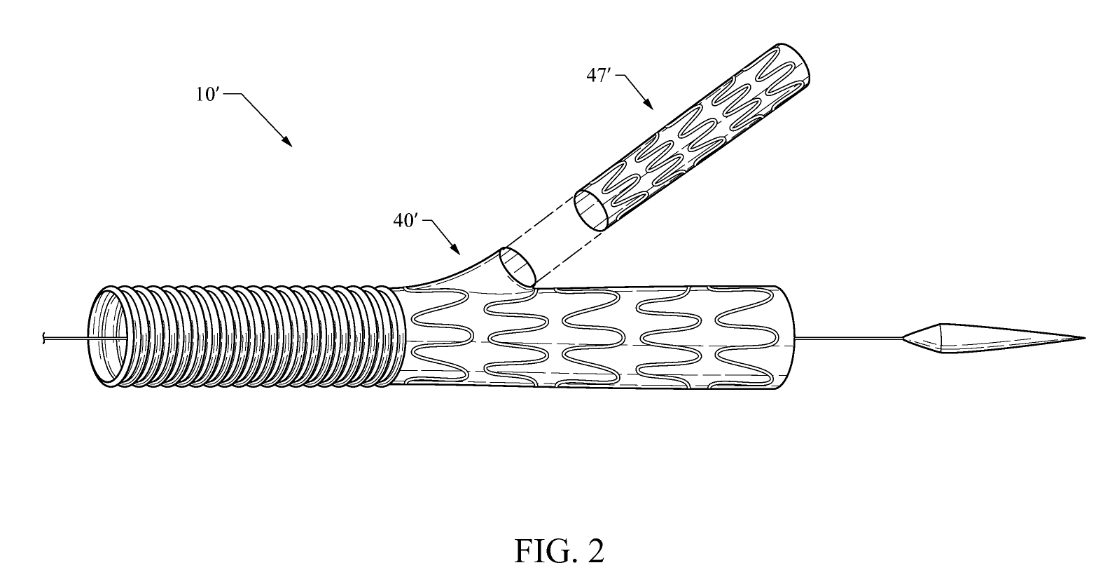

FIG. 2 illustrates another embodiment of a prosthesis 10′. The prosthesis 10′ may be substantially identical to the prosthesis 10 with the exception of the branch. The branch of the distal graft portion of the prosthesis 10′ may be configured as an extension 40′ as shown in FIG. 2. The extension 40′ may be relatively short as compared to the branch 40 of the prosthesis 10 and may be configured to receive a branch prosthesis 47. The branch prosthesis 47 may be configured as a stent graft. The branch prosthesis 47 may be deployed within the extension 40′ using any suitable technique (e.g., endovascular techniques) and may be configured to extend, for example, into a left subclavian artery of a patient as described below with reference to the branch 40. Any discussion in this disclosure regarding the prosthesis 10 may be generally applicable to the prosthesis 10′.

Returning to FIG. 1, the branch 40 may be attached to the main body 21 by sutures, wire, staples, clips, bonding agents, or other methods that may be used to achieve a secure attachment. For example, the branch 40 may be attached to the main body 21 by any method described in U.S. Patent Application Pub. No. 2006/0095118 to Hartley which is incorporated by reference herein in its entirety. The branch 40 may be attached to the graft body 22 and/or the support structure 30 of the main body 21. Preferably, the graft body 45 of the branch 40 may be attached to the graft body 22 of the main body 21 to form a fluid-tight seal. For example, the graft body 45 of the branch 40 may be stitched to the graft body 22 of the main body 21. An aperture may be formed in the graft body 22 of the main body 21. The aperture may be aligned with the first end opening 42 of the branch 40 to enable fluid communication between the main lumen 26 and the branch lumen 49 through the aperture. In this manner, the distal graft portion 20 of the prosthesis 10 may be configured to serve as a conduit for blood to flow through the main body and branch lumens 26, 49 from the proximal end 27 of the main body 21 to the distal end 28 of the main body 21 and the second end 43 of the branch 40.

The proximal graft portion 60 of the prosthesis 10 may include a substantially tubular graft body 62 having an inner surface 63 and an outer surface 64. The graft body 62 may be configured as a generally tubular member having a substantially cylindrical shape. The inner surface 63 of the graft body 62 may define a main lumen 66 extending longitudinally within the graft body between a proximal end 67 and a distal end 68 of the proximal graft portion 60. The main lumen 66 may be suitable for passing fluid such as, for example, blood therethrough. The proximal graft portion 60 may not include a support structure such as a stent. In other words, the proximal graft portion 60 may be devoid of stents or unstented as shown in FIG. 1.

The graft body 62 of the proximal graft portion 60 may be corrugated or crimped to increase flexibility and/or decrease the risk of kinking. Such corrugation or crimping also may help to promote setting of the proximal graft portion in a curved shape. Suitable crimps and methods for crimping the graft body 62 are described in U.S. Pat. No. 7,407,509 and U.S. Patent Application Pub. No. 2005/0113905 to Greenberg et al., which are incorporated by reference herein in their entirety.

The proximal graft portion 60 may extend proximally from the distal graft portion 20. To that end, the distal end 68 of the proximal graft portion 60 may be attached to the proximal end 27 of the distal graft portion 20. The graft body 62 of the proximal graft portion 60 and the graft body 22 of the distal graft portion 20 may be formed integrally with one another as a unitary structure. In other words, the graft body 62 and the graft body 22 may be formed together from a single piece of graft material. Alternatively, the graft body 62 of the proximal graft portion 60 and the graft body 22 of the distal graft portion 20 may be formed separately and attached to one another to form the prosthesis 10. The graft body 62 and the graft body 22 may be attached to one another by any means including, for example, those described above with reference to the branch 40 and the main body 21 of the distal graft portion 20. The lumen 66 of the proximal graft portion 60 may be in fluid communication with the main lumen 26 of the distal graft portion 20 to create a substantially continuous flow path between the proximal end 67 of the proximal graft portion and the distal end 28 of the distal graft portion.

The prosthesis 10 may be sized and shaped for placement within the vasculature of a patient as further described below. The preferred size and shape of the prosthesis 10 depends on the anatomy in which it is to be implanted. Physiological variables, deployment characteristics, and other factors also may contribute to the determination of a proper size and shape of the prosthesis 10. For example, the prosthesis 10 may have a size and shape suitable for placement in the ascending aorta, aortic arch, and/or descending aorta. To that end, the proximal graft portion 60 may be configured for placement within the ascending aorta and extending into the aortic arch. The distal graft portion 20 may be configured for placement within the aortic arch and extending into the descending aorta. The branch 40 may be configured to extend from the aortic arch into the left subclavian artery. The main body 21 of the distal graft portion 20 may have a diameter, for example, ranging from about 10 mm to about 46 mm. The diameter of the main body 21 may be constant along the length thereof. Alternatively, the main body 21 may be tapered such that the diameter of the main body may vary along the length thereof. The branch 40 may have a diameter, for example, ranging from about 8 mm to about 14 mm. The diameter of the branch 40 may be constant along the length thereof. Alternatively, the branch 40 may be tapered such that the diameter of the branch may vary along the length thereof. The main body 61 of the proximal graft portion 60 may have a diameter, for example, ranging from about 20 mm to about 40 mm. The diameter of the main body 61 may be constant along the length thereof. Alternatively, the main body 61 may be tapered such that the diameter of the main body may vary along the length thereof. In one example, main body 21 of the distal graft portion 20 may have a diameter that is different (e.g., larger or smaller) than the diameter of the main body 61 of the proximal graft portion 60. In this example, the distal end of the main body 61 and/or the proximal end of the main body 21 may be tapered so that the distal graft portion 20 and the proximal graft portion 60 may be attached or formed integrally with one another as described above. The prosthesis 10 may be deployed in combination with various other prostheses to effectively bridge an aneurysmal portion of the vasculature.

It is further contemplated that a distal graft portion of a prosthesis may have multiple branches extending from a main body. For example, the prosthesis may have two, three, or more branches extending from the main body. The various branches may be attached to the main body at different longitudinal and/or circumferential positions with respect to the main body. In this manner, the branches may be configured to extend into the left subclavian, left common carotid, and/or innominate arteries. The prosthesis also may be configured for placement at various other positions within the vasculature of the patient.

The graft bodies 22, 45, 62 may be made of any material known in the art. The graft bodies may be made of the same or different materials. Preferably, the graft bodies may be formed from a biocompatible material that is substantially non-toxic in the in vivo environment of its intended use and substantially unrejected by the patient's physiological system (i.e., is non-antigenic). For example, the graft bodies may be made of an expanded polytetrafluoroethylene (ePTFE), polytetrafluoroethylene, silicone, polyurethane, polyamide (nylon), polyethylene, polypropylene, polyaramids, polyacrylonitrile, and cellulose, as well as other flexible biocompatible materials. The graft bodies also can be made of known fabric graft materials such as woven polyester such as DACRON® from Invista (Wichita, Kans.), polyetherurethanes such as THORALON® from Thoratec Corporation (Pleasanton, Calif.), or polyethylene such as an ultra-high molecular weight polyethylene (UHMwPE) such as DYNEEMA® from DSM Dyneema LLC (Stanley, N.C.). In addition, materials that are not inherently biocompatible may be subjected to surface modifications to render the materials biocompatible. Examples of surface modifications include, for example, graft polymerization of biocompatible polymers on the surface, coating of the surface with a crosslinked biocompatible polymer, chemical modification with biocompatible functional groups, and immobilization of a compatibilizing agent such as heparin or other biocompatible substances. Thus, any fibrous material having sufficient strength to survive in the in vivo environment may be used to form a textile graft, provided the final textile is biocompatible.

The graft bodies also may include a bioremodelable material such as reconstituted or naturally-derived collagenous materials, extracellular matrix (ECM) material, submucosa, renal capsule membrane, dermal collagen, dura mater, pericardium, fascia lata, serosa, peritoneum or basement membrane layers, or intestinal submucosa, including small intestinal submucosa (SIS), stomach submucosa, urinary bladder submucosa, and uterine submucosa. One non-limiting example of a suitable remodelable material is SURGISIS® BIODESIGN™ from Cook Medical (Bloomington, Ind.). Another suitable remodelable material is the graft prosthesis material described in U.S. Pat. No. 6,206,931 to Cook et al., which is incorporated herein by reference in its entirety. The graft bodies also may be made of any of the materials described in U.S. Pat. No. 7,407,509 to Greenberg et al. or U.S. Patent Application Pub. No. 2009/0171451 to Kuppurathanam et al., which are incorporated herein by reference in their entirety.

The support structures 30, 50 and/or various portions thereof may be stents having any stent pattern known in the art. The stents may be balloon expandable. Preferably, the stents may be self-expandable. The stents can maintain the patency of the prosthesis and ensure adequate sealing against the surrounding vascular tissue. Some of the goals for stent design and placement, whether internal or external, may include preventing metal-to-metal contact points, preventing contact between two different types of alloys, and minimizing micromotion. Preferably, stent sizing, spacing, and design may be determined so that there is no stent-to-stent contact even in tortuous anatomy. Stents preferably may be placed to maximize prosthesis flexibility while maintaining patency, as well as reducing material wear and stent fatigue. Furthermore, it is preferable that the stents do not interfere with the branch, that they minimize the potential for galvanic corrosion, and ensure adequate joint stability. Stent amplitude, spacing, and stagger preferably may be optimized for each prosthesis design. Any of the stents mentioned herein may have barbs and/or other anchoring members to help decrease prosthesis migration.

One example of a stent pattern is the Z-stent or Gianturco stent design. Each Z-stent may include a series of substantially straight segments, or struts, interconnected by a series of bent segments, or bends. The bent segments may include acute bends or apices. The Z-stents may be arranged in a zigzag configuration in which the straight segments are set at angles relative to one another and are connected by the bent segments. This design provides both significant radial force as well as some longitudinal support. In tortuous anatomy or branches, it may be preferable to use alternative stents or modifications to the Z-stent design to avoid stent-to-stent contact. Alternative stents may include, for example, annular or helical stents. Furthermore, in complex anatomical situations, external stents have the potential to become intertwined with the wires and other devices utilized to ensure branch vessel access, sealing, and fixation. In some instances, it may be desirable to affix some of the stents to the internal surface of the prosthesis.

The stents described herein may be made from any suitable material known in the art. In one example, the stents may be made from standard medical grade stainless steel and may be soldered using silver standard solder (0 lead/0 tin). In other examples, the stents may be made from a metallic material selected from stainless steel, silver, platinum, palladium, gold, titanium, tantalum, iridium, tungsten, cobalt, chromium, cobalt-chromium alloy 1058, cobalt-based 35N alloy, nickel-based alloy 625, a molybdenum alloy, a molybdenum alloy including about 0.4% to about 0.8% of lanthanum oxide (Li₂O₃), and a nickel-titanium alloy, such as nitinol, or other suitable materials known in the art. The stents also may be made from nitinol or other shape-memory metal. Moreover, the stents may be configured in a variety of ways to provide a suitable intraluminal support structure. For example, one or more stents may be made from a woven wire structure, a laser-cut cannula, individual interconnected rings, or another pattern or design.

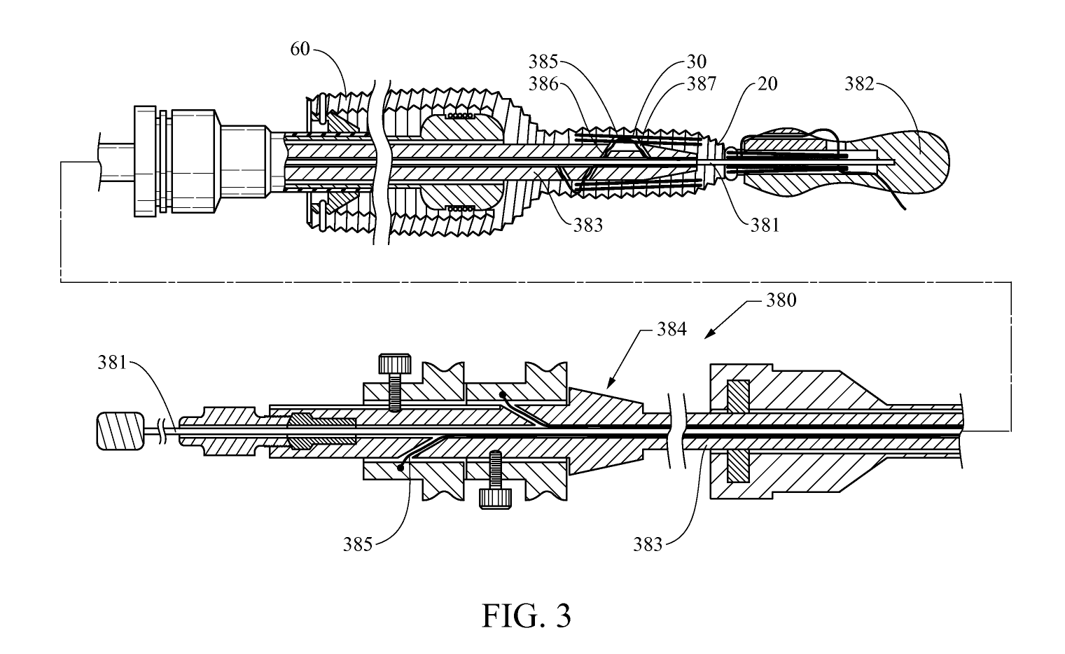

Referring now to FIGS. 3-9, exemplary systems and method steps for using the prosthesis of FIG. 1 to treat a condition in the area of a patient's ascending aorta, aortic arch, and/or descending aorta are shown and described.

The prosthesis 10 may be compressed into a delivery configuration and mounted onto a deployment device such as an introducer. Any type of deployment device suitable for deploying a branched stent graft may be used. For example, suitable deployment devices may include those described in U.S. Pat. Nos. 7,488,344 and 7,537,606 to Hartley et al.; and U.S. Patent Application Pub. No. 2004/0193244 to Hartley et al.; all of which are incorporated by reference herein in their entirety. The operator may directly manipulate the introducer, which may provide the operator with a relatively high degree of control during the procedure. Further, such deployment devices may be compact and may have a relatively uniform, low-diameter radial profile, allowing for atraumatic access and delivery.

FIG. 3 illustrates one example of an introducer 380. The introducer 380 may include a central catheter 381 extending from a proximal end of the introducer to a tip 382 at a distal end of the introducer. In use, the introducer may be introduced into the aorta from an incision in the patient's chest and advanced distally as further described below. In such a procedure, the proximal portion of the introducer may correspond generally to the proximal end of the prosthesis and the distal portion of the introducer may correspond generally to the distal end of the prosthesis. A deployment catheter 383 may surround the central catheter 381. The deployment catheter 383 may be moved longitudinally with respect to the central catheter 381 and may be locked into position with respect to the central catheter in known fashion by means of a pin vice arrangement at the proximal end of a handle 384. The handle 384 may be positioned at the proximal end of the deployment catheter 383 and may remain outside of the patient during use of the introducer.

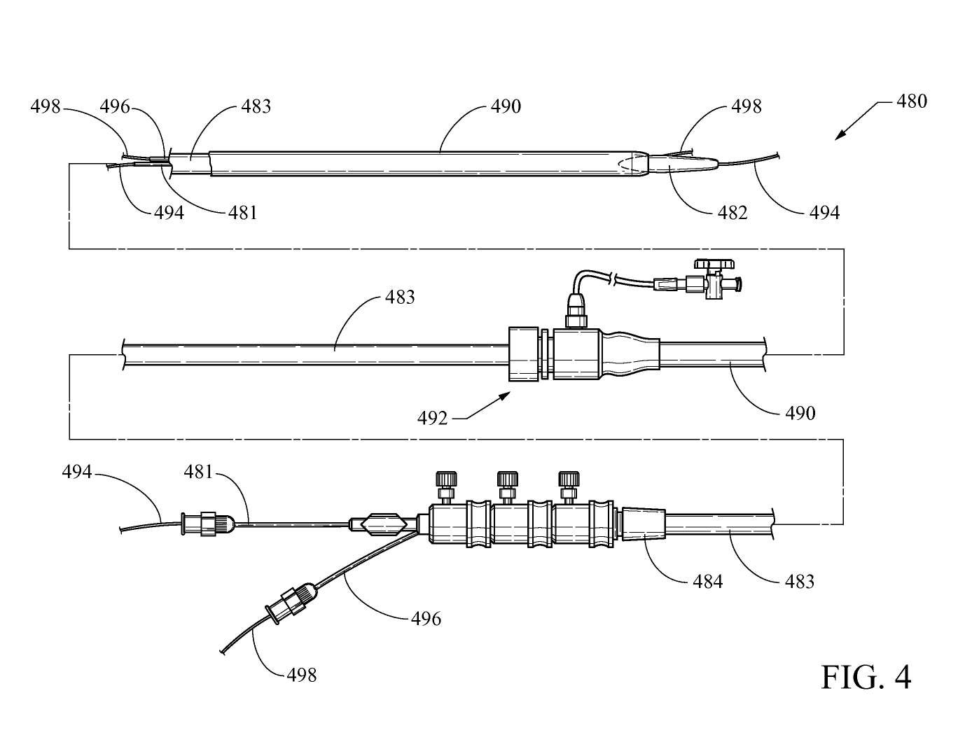

The prosthesis 10 may be delivered using any other suitable introducer. FIG. 4 illustrates another example of an introducer 480. The introducer 480 may be configured substantially as described in U.S Patent Application Pub. No. 2009/0254170 to Hartley et al., which is incorporated by reference herein in its entirety. For example, the introducer 480 may include a central catheter 481 received within a lumen of a deployment catheter 483. A sheath 490 may extend distally from a sheath manipulator 492 to cover at least a portion of the deployment catheter 483. A nose cone 482 may be attached to the distal end of the central catheter 481, and a handle 484 may be positioned near the proximal end of the central catheter. A guide wire 494 may extend within the central catheter 481. The introducer 480 also may include an auxiliary catheter 496 with an auxiliary guide wire 498 received therein. The auxiliary guide wire 498 may be positioned within the branch 40 of the prosthesis 10 to cannulate a branch vessel in any known manner. A set of trigger wire release arrangements also may be mounted on the handle 484 to manipulate a series of trigger wires as further described below.

To load the prosthesis 10 in an introducer (e.g., the introducer 380 or the introducer 480), the distal end of the introducer may be inserted distally into the lumen 66 of the proximal graft portion 60 and advanced distally within the prosthesis 10 until the distal end of the introducer exits the distal end 28 of the distal graft portion 20. The distal graft portion 20 of the prosthesis 10 may be compressed around the central catheter near the distal end of the introducer as shown in FIG. 3. In some examples, such as when using the introducer 480 of FIG. 4, the sheath may be positioned around the distal graft portion 20 of the prosthesis 10 to retain the distal graft portion in a compressed, delivery configuration.

When the prosthesis 10 is loaded on an introducer, the proximal graft portion 60 of the prosthesis 10 may be positioned around a portion of the central catheter proximal of the distal graft portion 20. The proximal graft portion 60 may be gathered, folded, or otherwise arranged in a reduced diameter and/or a reduced length configuration around the central catheter. In some examples, the proximal graft portion 60 of the prosthesis 10 may be positioned within the sheath of an introducer.

As shown in FIG. 3, the prosthesis 10 may be releasably attached to the introducer 380 to temporarily fix the prosthesis in place relative to the introducer until positioned as desired within the patient's body. For example, one or more trigger wires may engage a portion of the prosthesis 10 and may be releasably attached to the introducer 380 as further described below.

When mounted on the delivery device, at least the distal graft portion 20 of the prosthesis 10 may be compressed into a reduced diameter delivery configuration. In other words, the support structure 30 of the main body 21 may be compressed. The support structure 50 of the branch 40 also may be compressed. In one example, the prosthesis 10 may be radially compressed over the central catheter 381 of the introducer 380. The main body 21 and/or the branch 40 of the distal graft portion 20 may be retained in the delivery configuration by any suitable means. Suitable means for retaining the distal graft portion 20 in the delivery configuration may include those described in U.S. Pat. No. 5,456,713 to Chuter and U.S. Patent Application Pub. Nos. 2006/0004433 to Greenberg et al.; 2007/0142896 to Anderson et al.; 2008/0114438 to Hartley et al.; and 2008/0294234 to Hartley et al., which are incorporated by reference herein in their entirety.

In one example, a trigger wire 385 may extend along a length of the delivery catheter. The trigger wire 385 may extend between the central catheter 381 and the deployment catheter 383 within the lumen of the deployment catheter from the handle 384 to the distal end of the introducer 380. The trigger wire 385 may extend through a side aperture 386 in the deployment catheter 383 to exit the deployment catheter to engage and retain a stent 30 of the distal graft portion 20 in a compressed configuration. The trigger wire 385 may directly engage the stent 30 to retain the stent in close proximity to the introducer 380 (i.e., in the compressed configuration). The trigger wire 385 also may engage one or more reducing members such as diameter reducing ties or mooring loops to retain the stent 30 in the compressed configuration. After the trigger wire 385 engages the stent 30, the trigger wire may re-enter another side aperture 387 in the deployment catheter and extend further distally out of the distal end 28 of the distal graft portion 20 of the prosthesis 10. The trigger wire 385 may be received within one or more apertures of the tip 382 to retain the distal end of the trigger wire and to prevent the trigger wire from fouling with other objects during deployment of the prosthesis 10. The trigger wire 385 may be held in tension between the handle 384 and the tip 382 to oppose the outward radial force of the support structure 30 to retain at least a portion of the distal graft portion 20 in the compressed configuration. Releasing the trigger wire 385 may enable expansion of the distal graft portion 20 as further described below.

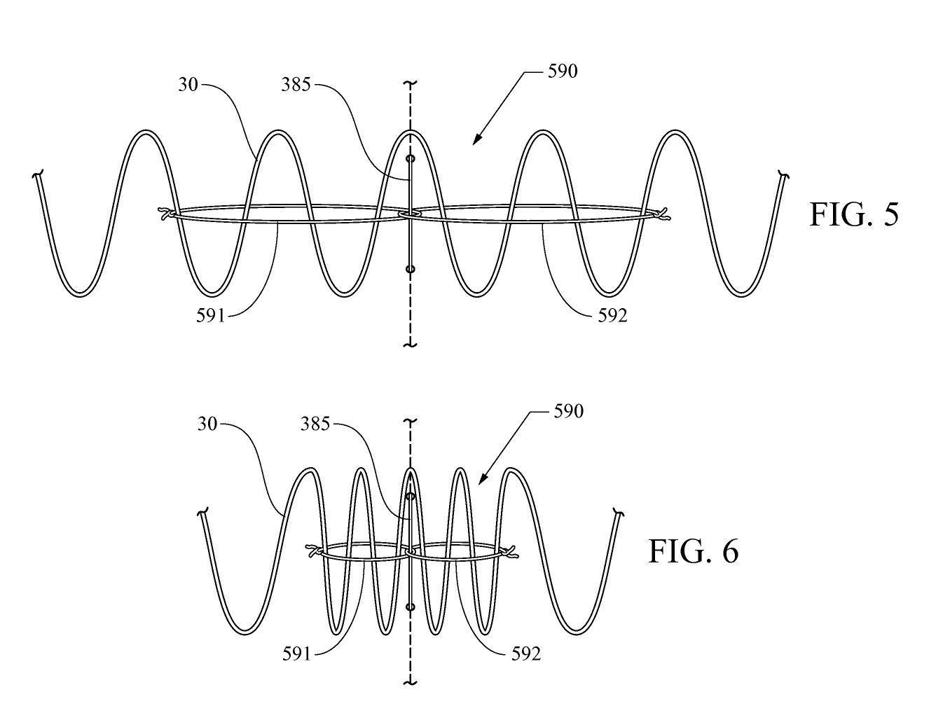

FIGS. 5-6 illustrate the application of one example of a diameter reducing tie 590 that may be applied to the prosthesis 10. A first thread 591 may be passed around the trigger wire 385 so that opposing end portions of the first thread are extended out to one side of the trigger wire. Each of the end portions of the first thread 591 may pass over a series of intermediate struts of the stent 30. One of the end portions then may pass over a target strut and the other end portion may pass under the target strut, as shown in FIG. 5, so that the first thread 591 forms a loop around the trigger wire 385 and the target strut. The two end portions of the first thread 591 may be tied in a knot, which may be pulled tight as shown in FIG. 6 so that the intermediate struts between the trigger wire 385 and the knot are pulled closer together. Excess portions of the first thread 491 extending beyond the knot may be trimmed as shown in FIG. 6. A second thread 592 may be applied in a similar manner. The thread 592 may be passed around the trigger wire 385 or around the two strands of the first thread 591 and across the trigger wire. Opposing end portions of the second thread 592 may be extended out to the other side of the trigger wire, opposite the first thread 591, over a series of intermediate struts, and looped around a target strut as shown in FIG. 5. The two end portions of the second thread 592 may be tied in a knot, which may be pulled tight as shown in FIG. 6 so that the intermediate struts between the trigger wire 385 and the knot are pulled closer together. In this manner, the circumference of the stent 30 may be restrained so that the diameter of at least a portion of the distal graft portion 20 of the prosthesis 10 may remain reduced for delivery within the patient.

The trigger wire 385 also may be stitched longitudinally along at least a portion of the prosthesis 10 (e.g., the main body 21 of the distal graft portion 20) to engage the diameter reducing ties or mooring loops applied to the prosthesis. Stitching the trigger wire into the graft material of the prosthesis may render the deployment catheter unnecessary. In other words, one or more trigger wires may be stitched into the graft material of the prosthesis so that positioning the trigger wires between the central catheter and the deployment catheter may not be required, and the deployment catheter may be omitted from the introducer.

Although the trigger wire 385 has been described as retaining only a single stent of the distal graft portion 20 of the prosthesis 10 in the compressed configuration, the trigger wire may engage any or all of the stents along a length of the main body 21 of the distal graft portion in similar fashion. For example, multiple pairs of apertures may be provided in the deployment catheter 383 so that the trigger wire 385 may exit and re-enter the deployment catheter at multiple points along a length thereof. The apertures may be aligned with the stents 30 so that the trigger wire 385 may engage a stent (or a diameter reducing tie or mooring loop) adjacent each pair of apertures. Multiple trigger wires also may be provided. Each trigger wire may engage one or more stents, diameter reducing ties, or mooring loops. In this manner, the main body 21 of the distal graft portion 20 of the prosthesis 10 may be retained in the compressed configuration along substantially an entire length thereof.

Additional trigger wires, diameter reducing ties, and/or mooring loops may be provided along a length of the branch 40 of the distal graft portion 20 of the prosthesis 10. Separate trigger wires may be provided for the main body 21 and/or the branch 40. In this manner, the main body 21 and the branch 40 may be selectively expanded as desired by the operator. To deploy the prosthesis 10, or the distal graft portion 20 thereof, the operator may pull or retract one or more trigger wires, thereby releasing the trigger wires from the stents, diameter reducing ties, and/or mooring loops to enable outward expansion of at least a portion of the distal graft portion 20 of the prosthesis 10.

Any other suitable delivery device may be used to deploy the prosthesis 10. For example, the prosthesis 10 or a portion of the prosthesis (e.g., the distal graft portion 20) may be received within a conventional peel-away sheath. The prosthesis 10 within the peel-away sheath may be loaded onto a positioner and introduced within a body vessel using any suitable method. With the distal graft portion 20 of the prosthesis 10 in a desired position within the body vessel, the peel-away sheath may be removed to enable expansion of the distal graft portion of the prosthesis.

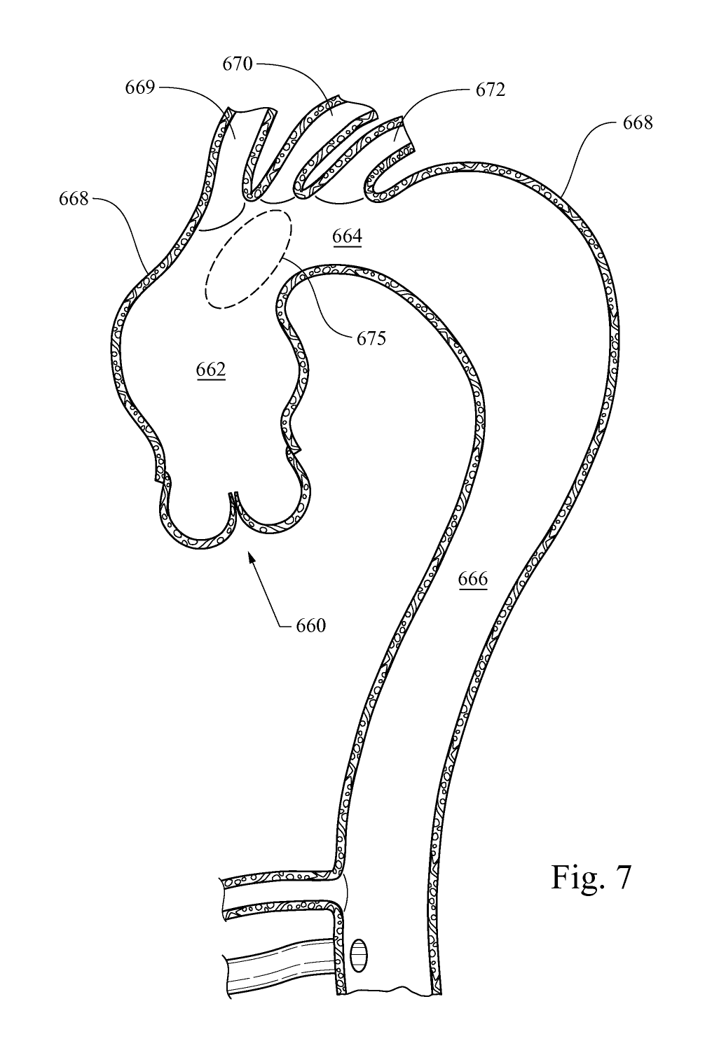

FIG. 7 shows a schematic view of a thoracic arch region of an aorta of a patient. The aorta extends from an aortic valve 660 of the patient via the ascending aorta 662 to the thoracic arch 664 before proceeding down the descending aorta 666. An aneurysm 668 has been depicted in the ascending aorta as well as adjacent the thoracic arch 664 in the descending aorta 666. In the arch region 664, major arteries including the innominate artery 669, the left common carotid artery 670, and the subclavian artery 672 branch off from the aorta. Any deployment of a prosthesis into the aorta preferably may maintain blood perfusion to these arteries.

After cooling the patient's body temperature, exposing the aortic arch through a sternotomy, and performing cardiopulmonary bypass or systemic circulatory arrest, an incision 675 may be made in the side of the thoracic arch 664. As shown in FIG. 7, the incision may extend from a portion of the thoracic arch 664 adjacent the ostia of the innominate artery 669 and left common carotid artery 670 to the distal portion of the ascending aorta 662. However, because the distal graft portion 20 of the prosthesis 10 may be deployed using endovascular techniques, as further described below, it may be unnecessary to expose the ostium of the left subclavian artery 672 or the proximal portion of the descending aorta 666.

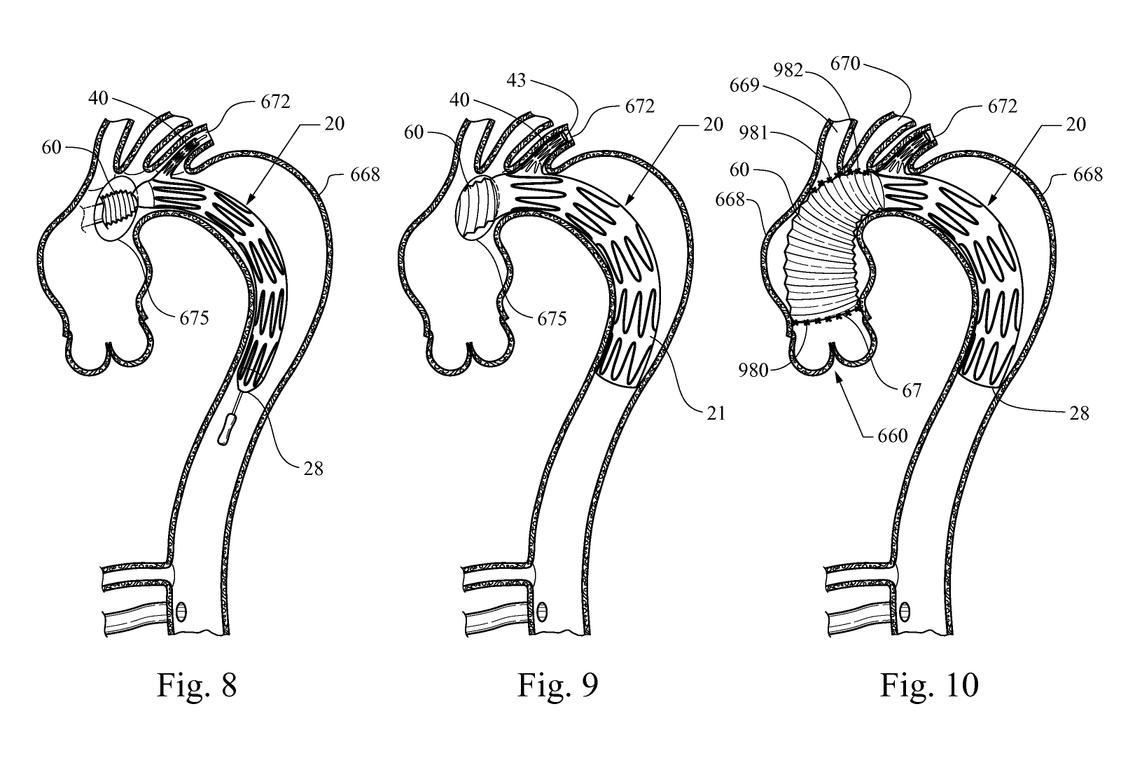

The distal graft portion 20 of the prosthesis 10 may be deployed using endovascular techniques. The prosthesis 10 may be placed transapically. For example the distal graft portion 20 of the prosthesis 10, in the delivery configuration and mounted on the distal end of the introducer 380, may be inserted through the incision 675, through the aortic arch 664, and into the descending aorta 666. A proximal portion of the introducer (e.g., the handle of the introducer) may remain external to the aorta throughout deployment of the prosthesis 10. With the distal graft portion 20 of the prosthesis disposed within the aorta, a delivery device such as an introducer, guide wire, catheter, or dilator may be inserted into the prosthesis 10 to cannulate the left subclavian artery. For example, the delivery device may be inserted into the prosthesis 10 from the proximal end 67 of the proximal graft portion 60. The delivery device may be advanced distally through the lumen 66 of the proximal graft portion 60 and into the lumen 26 of the main body 21 of the distal graft portion 20. The delivery device may be further advanced through the lumen 49 of the branch 40 to exit the second end 43 of the branch. A tip of the delivery device may be inserted through the ostium of the left subclavian artery 672 to cannulate the left subclavian artery. The introducer 380 and the prosthesis 10 may be advanced distally within the descending aorta 666 until the distal end 28 of the distal graft portion 20 is positioned distal to the aneurysmal region 668 as shown in FIG. 8. In this position, the branch 40 of the distal graft portion 20 may be generally aligned in the vicinity of the ostium of the left subclavian artery 672. The proximal graft portion 60 of the prosthesis 10 may extend through the incision 675 to remain substantially external of the aorta. The proximal graft portion 60 may remain accessible for direct manipulation by the physician.

The distal graft portion 20 of the prosthesis 10 may be expanded as shown in FIG. 9. The main body 21 may be expanded by, for example, retracting a trigger wire to disengage the stents of the main body or to release the diameter reducing ties or mooring loops retaining the main body in the compressed configuration. In other examples, the main body 21 may be expanded by retracting a sheath of the introducer in a known manner. Upon expansion of the main body 21, the branch 40 of the distal graft portion 20 may move distally along the delivery device positioned within the left subclavian artery 672 such that the second end 43 of the branch 40 may be moved to a position within the left subclavian artery. The branch 40 may be expanded by, for example, retracting a trigger wire to disengage the stents of the branch or to release the diameter reducing ties or mooring loops retaining the branch in the compressed configuration. The deployment device and/or delivery device may be retracted and removed from the patient's body following deployment of the distal graft portion 20 of the prosthesis 10.

Following deployment of the distal graft portion 20, the proximal graft portion 60 of the prosthesis 10 may be placed into the aorta using surgical techniques. For example, the proximal graft portion 60 may be manually introduced into the incision 675 in the thoracic arch 664 and directed into the ascending aorta 662 toward the aortic valve 660 as shown in FIG. 10. The proximal end 67 of the proximal graft portion 60 may be sutured circumferentially at suture 980 around the aortic valve 660 so that blood may flow out of the valve and into the prosthesis 10. An incision may be made through the graft material of the proximal graft portion 60 adjacent the ostium of the innominate artery 669. The proximal graft portion 60 may be sutured circumferentially at suture 981 around the ostium of the innominate artery 669 so that blood may flow out of the prosthesis 10 and into the innominate artery. Another incision may be made through the proximal graft portion 60 adjacent the ostium of the left common carotid artery 670. The proximal graft portion 60 may be sutured circumferentially at suture 982 around the ostium of the left common carotid artery 670 so that blood may flow out of the prosthesis 10 and into the left common carotid artery. In this manner, a substantially continuous flow pathway may be provided from the proximal end 67 of the proximal graft portion 60 of the prosthesis 10 into the innominate artery 669, the left common carotid artery 670, and the left subclavian artery 672, as well as to the distal end 28 of the distal graft portion 20 of the prosthesis 10. Any type of surgical fastening may be used instead of or in addition to the sutures described above. The proximal graft portion may be surgically fastened to the aorta by, for example, sutures, wire, staples, clips, bonding agents, or other methods that may be used to achieve a secure attachment. The aneurysmal portions 668 of the aorta also may be substantially excluded by the prosthesis 10. The incision 675 and the chest cavity may then be closed.

Both endovascular and surgical techniques may be employed to perform the hybrid procedure described above. Such a hybrid procedure may have several advantages. For example, the use of endovascular techniques to deploy the distal graft portion of the prosthesis may make direct access to the ostium of the left subclavian artery or the proximal portion of the descending aorta by the physician unnecessary. Such direct access may be unnecessary because the procedure may not require sutures to be made around the ostium of the left subclavian artery or the proximal portion of the descending aorta. Direct access also may be unnecessary because the branch of the distal graft portion of the prosthesis may be inserted and deployed within the left subclavian artery from the proximal end of the proximal graft portion of the prosthesis. In other words, the physician may guide the branch into place from outside of the aorta using endovascular techniques and without directly accessing the left subclavian artery. The procedure, therefore, may be performed through a relatively small incision near the distal portion of the ascending aorta and/or the proximal end of the thoracic arch. The ostium of the left subclavian artery and the proximal portion of the descending aorta may remain substantially directly inaccessible throughout the procedure. In other words, because of the size and position of the incision, the ostium of the left subclavian artery and the proximal portion of the descending aorta may remain unexposed throughout the procedure. This may result in a less invasive procedure that may be performed on patients who may be intolerant of more invasive procedures such as Bentall or total arch replacement procedures.

The hybrid procedure described above also may be performed in a relatively short amount of time as compared to other procedures such as Bentall or total arch replacement procedures. For example, a physician may have difficulty reaching the ostium of the left subclavian artery to suture a graft thereto, as may be required by a procedure such as a total arch replacement procedure. A physician also may have difficulty reaching the proximal portion of the descending aorta. Such difficulty may be a result of the anatomy of the patient. For example, the position of the aorta may transition from an anterior position within the patient's body at the proximal portion of the thoracic arch to a posterior position at the proximal portion of the descending aorta. In other words, the proximal portion of the descending aorta may be positioned deeper within the patient's chest than the proximal portion of the thoracic arch. This may make direct access to the distal portion of the thoracic arch (e.g., the left subclavian artery) and/or the proximal portion of the descending aorta difficult for the physician. Because the distal graft portion of the prosthesis may be positioned without directly accessing the distal portion of the thoracic arch or the proximal portion of the descending aorta, direct access to the portions of the aorta that may be more difficult to reach may be unnecessary. Thus, the hybrid procedure may be performed in a relatively short amount of time as compared to other surgical procedures.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A hybrid prosthesis, comprising:
a distal graft portion including a tubular main body comprising a biocompatible material and a support structure, the distal graft portion having proximal and distal ends and a main lumen extending therebetween, at least one tubular branch extending from the main body of the distal graft portion and having a first end, a second end, and a branch lumen extending therebetween, the branch lumen being in fluid communication with the main lumen; and
a proximal graft portion including a tubular body comprising a biocompatible material, the body of the proximal graft portion being unstented and having a proximal end, a distal end, and a lumen extending therebetween;
wherein the distal end of the proximal graft portion is attached to the proximal end of the distal graft portion and the lumen of the proximal graft portion is in fluid communication with the main lumen of the distal graft portion.

2. The hybrid prosthesis of claim 1, wherein the branch further comprises a support structure.

3. The hybrid prosthesis of claim 1, wherein at least a portion of the proximal graft portion is corrugated.

4. The hybrid prosthesis of claim 1, wherein the main body of the distal graft portion and the body of the proximal graft portion are formed from a single piece of graft material.

5. The hybrid prosthesis of claim 1, wherein the support structure of the distal graft portion comprises a plurality of stents.

6. The hybrid prosthesis of claim 5, wherein the plurality of stents comprises a distal stent positioned at the distal end of the distal graft portion.

7. The hybrid prosthesis of claim 5, wherein the plurality of stents comprises a proximal stent positioned adjacent an attachment point between the branch and the main body of the distal graft portion.

8. A system for the treatment of a vessel defect, the system comprising:
a hybrid prosthesis comprising:
a distal graft portion including a tubular main body comprising a support structure, the distal graft portion having a proximal end, a distal end, and a main lumen extending therebetween, at least one tubular branch extending from the main body of the distal graft portion and having a first end, a second end, and a branch lumen extending therebetween; and
a proximal graft portion extending from the distal graft portion, the proximal graft portion including a tubular body, the body of the proximal graft portion being unstented and having a proximal end, a distal end, and a lumen extending therebetween; and
an introducer comprising a central catheter;
wherein at least a portion of the distal graft portion of the hybrid prosthesis is retained in a compressed configuration on the central catheter of the introducer for endoluminal delivery to a site of the vessel defect.

9. The system of claim 8, further comprising at least one reducing member attached to the support structure of the distal graft portion to retain at least a portion of the distal graft portion in the compressed configuration.

10. The system of claim 9, further comprising at least one trigger wire threaded through a graft material of the distal graft portion and engaging the reducing member.

11. The system of claim 8, wherein the branch further comprises a support structure.

12. The system of claim 11, further comprising at least one reducing member attached to the support structure of the branch to retain at least a portion of the branch in the compressed configuration.

13. A method of treating a defect of a thoracic aorta of a patient, the method comprising:
making an incision in a thoracic arch of the patient, the incision being positioned proximal to an ostium of a left subclavian artery of the patient;
introducing a distal graft portion of a hybrid prosthesis through the incision and into the thoracic aorta;
endoluminally positioning a distal end of the distal graft portion within a descending aorta of the patient;
endoluminally positioning a branch of the distal graft portion within the left subclavian artery of the patient.

14. The method of claim 13, wherein the ostium of the left subclavian artery is substantially directly inaccessible from the incision in the thoracic arch.

15. The method of claim 13, wherein the ostium of the left subclavian artery remains substantially unexposed during introducing and positioning of the distal graft portion.

16. The method of claim 13, further comprising introducing a proximal graft portion of the hybrid prosthesis through the incision and into the thoracic aorta, the proximal graft portion extending proximally from the distal graft portion.

17. The method of claim 16, further comprising surgically fastening a proximal end of the proximal graft portion circumferentially around a proximal portion of an ascending aorta.

18. The method of claim 16, further comprising surgically fastening the proximal graft portion circumferentially around an ostium of an innominate artery and surgically fastening the proximal graft portion circumferentially around an ostium of a left common carotid artery.

19. The method of claim 13, further comprising allowing at least one self-expanding stent of the distal graft portion to expand within the thoracic aorta.

20. The method of claim 13, further comprising allowing at least one self-expanding stent of the branch to expand within the left subclavian artery.
